(19) United States
(12) Patent Application Publication
(10) Pub. No.: US 2023/0357816 A1
(43) Pub. Date: Nov. 9, 2023

(54) METHOD AND APPARATUS FOR THE UTILIZATION OF ZERO FIBER AND OTHER SIDE STREAMS

(71) Applicant: Eino Elias Hakalehto, Kuopio (FI)

(72) Inventor: Eino Elias Hakalehto, Kuopio (FI)

(21) Appl. No.: 18/021,819

(22) PCT Filed: Aug. 17, 2021
(86) PCT No.: PCT/FI2021/000008

§ 371 Date: Feb. 17, 2023

(30) Foreign Application Priority Data
Aug. 17, 2020 (FI) 20200056

(51) Int. Cl. C12P 39/00; C12P 7/56; C12P 7/18; C12M 1/00; C12M 1/40; C05F 17/20; C12P 7/6409

(52) U.S. Cl. CPC C12P 39/00; C12P 7/56; C12P 7/18; C12M 23/58; C12M 21/18; C05F 17/20; C12P 7/6409

(57) ABSTRACT

In the combined process for several biorefinery products obtained from a UMC (Undefined Mixed Culture) type of reaction it is possible to obtain biochemicals, energy gases, soil improvement etc. from a MPBU (Multipurpose Biorefinery Unit). The economically beneficial as well as environmentally sustainable results of the arrangement are demonstrated by the integrated process using two reactor systems with zero fiber for the production of lactate (in both the reactors pools 1 and 2). Additionally, mannitol can be produced in one of the reactor pools (number 2). It is possible to
a. combine the processes taking into account their biochemical characteristics,
b. produce gaseous substances for energy and industrial use,
c. obtain organic fertilizers which can be microbiologically upgraded
d. improve the adjustability for optimization of the various partial reactivities.

The chemical production occurs in two pools which advantageously are inoculated simultaneously.

### BACKGROUND

In traditional biotechnical processes using micro-organisms as biocatalysts it is usual to have single species or sometimes two species or strains of microbes performing the desired reaction. It is then believed to become more adjustable and controllable as well as more predictable one.

However, such microbiological processes seldom take place in natural habitats. Also, in the man-made processes there are often mixed inocula used as a seed. In such cases the microbes can strive for a balance with each other in a mixed culture. Such examples are the biogas production, composting of organic matter, and other bioprocesses taking place in a more ecosystem-like reaction environment. Such mixed substrate milieu can be found in the so called zero fiber waste which is produced in tens of thousands of tons annually as a side-stream of a big paper or cardboard factory. This fibrous material consists of the cellulosic molecules which are too short for e.g. paper-making. Zero fiber deposit have accumulated in the proximity of many forest industries, often onto the lake or sea bottom or an equivalent reservoir as a sediment (Hakalehto 2018a). Their processing into useful chemical substances has been developed for the production of such organic acids as lactic acid, for instance (Beckinghausen et al. 2019, Hakalehto 2020).

The maturation of any microbial community as a whole occurs in the industrial bioprocess setting in order to obtain the best possible results from one or more biochemical reactions. This also makes the contamination control usually much easier. However, sometimes it is also possible to get several end-products from the same reaction broth. This, in turn, sets up additional complications for the steering of the process and for the adjustment of its parameters. This is particularly true when the conditions have to be changed during the process in order to facilitate first the variable end-product formation of the hydrolysis reactions for obtaining appropriate raw material, and then for the switch to the actual production reaction or for avoiding any extensive end-product inhibition, catabolite repression or other biological regulation mechanism. It is also often adventageous to run a biorefinery process in an oscillating way, where the values for the key parameters change in a cyclic manner (Hakalehto et al. 2008).

The above-mentioned changes or transitions into more complex matrices of control parameters also produce more effective ways for controlling the process conditions provided that the process remains under control. The importance of this increased amount of adjustment tools with more flexibility will be emphasized in the ecosystem-type of bioreactor systems. Also, when the hydrolysis reactions are carried out in the same compartment or simultaneously with the product formation, this requires more sophisticated technologies for the measurement and control. If the production scale is growing, this demand for additional control further increases.

Many microbiological processes in the biorefineries (Hakalehto 2016a, b, 2018a, Den Boer et al. 2016, Schwede et al. 2017, Beckinghausen et al. 2019) have their counterpart or otherwise corresponding reactions in the digestive or alimentary tract (Hakalehto 2011, 2012, 2013). The concept of BIB (Bacteriological Intestinal Balance) has been developed for describing the internal strive of the digestive microbial ecosystem for establishing a balance. Some features of “habitat dominance by coalition” are discussed previously (Hakalehto 2018b). The basis of the self-control processes by interspecies dominance or succession can be observed between various members of some intestinal strains belonging to the family Enterobacteriaceae in the duodenum (Hakalehto et al. 2008), as well as by the interacting lactobacilli and clostridia in the large intestines (Hakalehto and Hänninen 2012).

The process of so-called Consolidated Bioprocessing (CBP) in practise means a bioprocess simultaneously using both common kinds of biocatalysts, namely the enzymes and the microbial strains. These biocatalysts are used for the processing of the organic polymeric raw materials as biocatalysts.

In fact, the industrial bioprocess broth somewhat resembles the intestinal chyme, both of them being non-aseptic bioreactor systems. This kind of process is advanced by a mixed microbial flora often called as UMC (Undefined Mixed Culture). As like in the alimentary tract, the corresponding process ecosystem is eventually seeking for a balance. Recognizing or identifying such balances as well as the steering or exploiting of them for the improved production of the desired chemicals or gases opens up new opportunities for microbial bioprocesses.

During the more or less fermentative i.e. anoxic processes, multitude of parameters influence the outcome of the reaction. Most of them are adjustable by the operator, which is a significant asset for the optimization of any biological production process. Such processes are characterized by Hakalehto et al. (2016), Hakalehto (2018a) and Jääskeläinen et al. (2016), for instance. The further improvement of these processes is also in the scope of this invention.

The above-mentioned parameters for the microbe process include:
temperature
pH
oxygen content
gassing
viscosity
mixing
pressure
fractionation
gradient formation
etc.

These adjustable traits essentially influence the outcome of the process. It can be altered also by adding some more strains with desired metabolic or regulatory characteristics into the process.

As it is often beneficial to have various gradients in the biorefinery process in the production broth; the establishment, enforcing and controlling of those gradients gives potential for steering up of the process. The processing goals can be promoted by intelligent adjustment, and also by dividing the process into sequences, phases, different compartments etc. Partial walls or semipermeable membranes can be used for this purpose. Various atmospheres have been created into various parts of the reactor by the above-mentioned means (Hakalehto 2008). According to this previous patent application aerobic and anoxic gas mixtures can be led into the different parts of the reactor. This forms gradients which can be beneficial for avoiding the regulatory mechanisms of the various members of the mixed microflora, for instance. Moreover, simultaneous accomplishment of different objectives becomes easier, such as the running of the CBP (Consolidated Bioprosessing) type of reactions (Hakalehto 2015a). This means the integration of enzymatic hydrolysis and the actual microbial process in the one and same reactor.

The common problems in the CBP unit includes different preferential or optional conditions for the enzymatic process and the microbe process. For the former one, the most important core parameters are:
concentration of the enzyme
temperature
pH
enzymatic activity
affinity of the enzyme toward the substrate(s)
duration of the effective time for enzymatic process
self-life of the enzyme molecules
enzyme sensitivity to disturbing factors
regulation of the enzymatic function

The final result of the hydrolysis process is a combination or a selection of these circumstances provided that the major substrate is not the limiting factor. The microbial process and its self-regulation are even more complicated sequences of events and successions of various strains or their different reactivities. Consequently, it is of crucial importance to identify the main reactions, their duration, optimal conditions and drivers, in order to integrate various processes (Hakalehto and Jääskeläinen 2017). Moreover, the use of mixed microflora in the bioprocess may further complicate the control, particularly in the case of multiple products whose manufacturing needs to be optimized simultaneously. Sometimes it can be beneficial to partially separate the various processes or their phases. In any case, various measurements and ways for monitoring the process and its phases are required.

For both the enzymatic process and the subsequent microbial process, the shape of the reactor as well as the reachability of the substrate by the biocatalyst are also important technical parameters when designing the reactor hardware for the CBP. The picture becomes even more complicated one, if several products are produced at the same time. Again we meet the limitations caused by the differing or conflicting requirements of the bioprocess within the one and same reactor system. This variation may occur regardless of the composition of the raw material, whether it is in a solution, broth, suspension, emulsion or on a solid phase. Likewise, the supposed movement or lack of movement, or the stability or instability or lability of the raw materials at any given time point does not eliminate the issue of finding difficulties in getting conditions right for the simultaneous catalytic processes of the successful bioreaction leading to useful results from chemical conversion of microbial metabolites into precious products, those metabolites being obtained from enzymatically degraded macromolecules.

### DESCRIPTION OF THE INVENTION

In biotechnical processes using biocatalysts, it is essential to implement the simultaneous planning strategies both for the growth and maintenance of the biocatalysts and for their reactions, as well as for the hardware design and adjustments of the bioreactors. The essential feature of this invention is to synchronize the biochemical and microbiological process with the design of the bioprocessing plant. These ideas have been tested in several pilot experiments, where both enzymes and microbial mixed cultures have been used in the pool-shaped reactors.

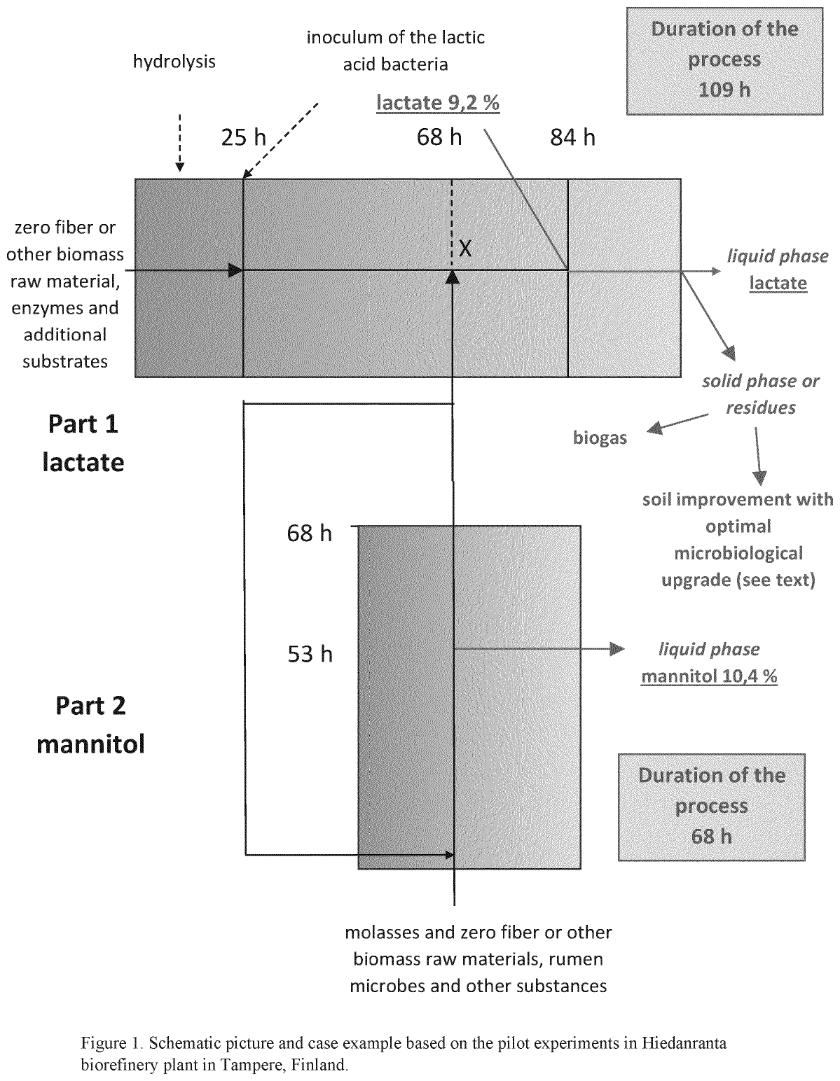

The pool construct allows the bioprocess fluid or broth or suspension to be moved forward while it is processed or adjusted. It is also easier to carry out the measurements of the process parameters alongside the progress within the reactors, or during the succession of the biochemical or microbiological reaction sequences. In the present invention, the pool shaped reactor system is illustrated in FIG. 1.

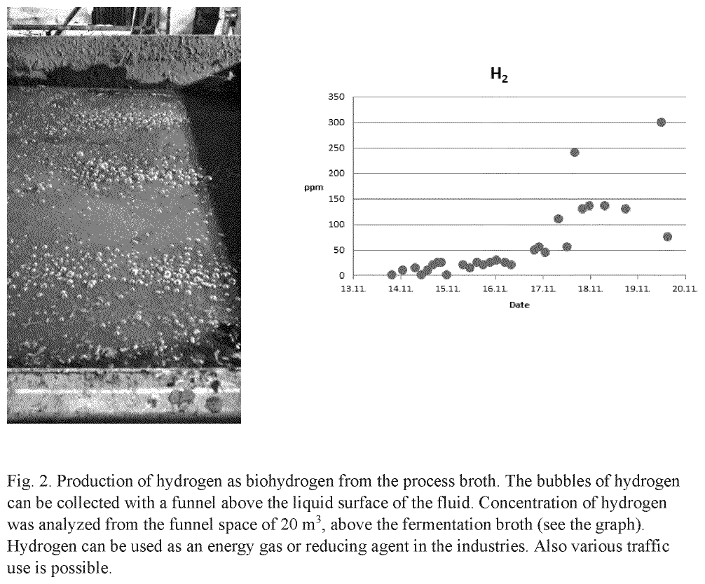

This flexibility of the process control is important, not only for the timely recovery of chemical products but also for the collection of gaseous substances. For example, the hydrogen gas can be formed during a specific phase of the process. If the oxygen content of the fermentation broth is low enough, this leads to the formation of butyrate and hydrogen (FIG. 2).

If the organic acids, such as lactate and propionate, are produced microbiologically from the slaughterhouse waste or from the potato industry waste, some members of the normal flora or the additional industrial strains of Clostridium pasteurianum could facilitate the formation of valeric acid (Den Boer et al. 2016; Schwede et al. 2017). The formation takes place as a consequence of the condensation reaction between lactate and propionate. Besides, the Clostridium pasteurianum strains or its closest relatives are strictly anaerobic bacteria which also produce hydrogen gas (H₂), and bind atmospheric nitrogen (N₂) in an autonomous fashion (Hakalehto 2016b). In fact, it has been proven out that the lactic acid bacteria can boost the onset of clostridial growth by their CO₂ production (Hakalehto and Hänninen 2012; Hakalehto 2015a). This could make it possible to combine the production of H₂ (for hytane gas mixture, for example) with the conversion of organic wastes into useful chemicals, such as organic acids (lactate, propionate, butyrate, acetate, valerate etc.). or 2,3-butanediol, butanol or ethanol, as well as with the production of sugar alcohols, such as mannitol, xylitol or sorbitol. This could lead to a biorefinery process, which could in the same or parallel units facilitate the production of
1. energy gases,
2. valuable chemicals, and
3. organic fertilizers or soil improvement agents,
   in the process unit with one or several industrial strains which could function together with the natural microflora derived from the side stream in question. The above-mentioned microbiological method to upgrade the residual fraction by autonomous Nitrogen-fixing bacteria could remarkably improve the economics of the zero fiber processing, or alternatively that of any other biomass processing multi-strain or CBP-type of bioprocess. This could produce huge savings in:
A. investment costs, as the production unit volumes go down,
B. energy efficiency, as the power source is within the process,
C. adjustments and control, which can be handled on the ecosystem level at best,
D. removing at least a part of the gate fees in the treatment of the residual fraction and by bringing an important economical value for it
   in the said manufacturing unit. The corresponding and required technologies could make it possible to learn to adjust the process for the numerous goods (gases, chemical commodities, fertilizers) according to the economic conditions and the demand in the market. Consequently, it is possible to build up a multipurpose biorefinery unit (MBPU) with low investment costs. It can obtain energy gases (hydrogen, methane, hytane) or electricity from the process itself. Such MBPU process, however, may also need clever partitioning of the process or unit operations.

Different organic materials and side streams can be produced in the MBPU. Besides the residual fractions of the forest, potato or slaughterhouse industries, also different agricultural or forestry wastes, as well as side streams of the sugar or brewing or fruit processing industries could be considered as potential raw materials. Since most of these raw materials consist of organic polymers, their hydrolysis is required. This could be carried out by acid or base, or by hot steam or water, or by some other physicochemical methods, as well as by enzymatic hydrolysis. In the latter kind of process, temperature changes could be utilized for improving the yield from the hydrolysis. The raw materials for the unit could include many other biomass sources besides the zero fiber, such as agricultural wastes, fruit waste, food industry wastes, sugar industry waste etc.

In addition to the two SCFA's (Small Chain Fatty Acids), lactate and propionate, it is possible to produce a third one, namely butyric acid (butyrate). This has been formed in the process utilizing the zero fiber and paunch as raw materials. It has been proven in our earlier studies that the CO₂ emitted by lactic acid bacteria provokes and speeds up the growth of butyric acid clostridia (Hakalehto and Hänninen 2012, Hakalehto 2015a).

Typically for the production of the SCFA's their formation is peaking in the anoxic conditions. If the pH is around 6.5, the main product of the mixed fermentation is often propionate, at the pH of 5.5 it is butyrate, and at the pH of 4.5 acetate. Lactate is converted into other SCFA's (Hakalehto 2015b). The production of propionate, for example, can also get performed by a food-grade micro-organism Propionibacterium acidipropionici, which is accepted for food production by EFSA (European Food Safety Association).

In order to carry out the CBP type of reaction, one has to support it or at least is obliged to suppose that the conditions for the enzymatic hydrolysis will remain allowable during the accompanying microbial process. In turn, the continuous hydrolysis keeps the conditions ideal for microbial metabolism as it limits such regulatory functions as feedback inhibition for itself. Therefore, it is beneficial for the outcome, productivity and yield of the process to ensure the incessant enzymatic function in the production broth, as well as the boosting up of the desired microbiological reaction in a mixed metabolism situation. In fact, the CBP process is often easier to be converted into a continuous process. However, if the products are mixed or variable ones, the processing plan may include several reactor, tanks or pools for various partial processes or phases.

In practise, the challenges of the CBP often relate to the diffusion reactions, which means in practise that gradients or different zones are easily formed into the process. This is more likely in the big units. On the other hand, these gradients could also be advantageous for the process outcome, productivity and yield, provided that the gradients can be controlled well enough.

Therefore, with the intention of
A. arranging suitable conditions throughout the reactor broth for both enzymatic hydrolysis and the microbial process, and
B. controlling the gradients related to various reactions the equipment and method according to the present invention offers means to exercise such activities when pursuing the multi-strain or CBP-type of reaction in a biorefinery or equivalent.

In a big production unit a pool-type of reactor if often advisable for the improved options of control and sequential process mode. According to the present invention, it is possible to monitor and measure such parameters as temperature, pH, turbidity, concentrations of various gases, conductivity, pO2, pCO2, impedance, viscosity, glucose or fructose content or any other parameter. These measurements can be taken from the process broth moving on by the rotors, propellers, liquid blows, screws, paddlewheels or equivalent. The measurement can be taken from any point of the process, and the result can be used for the adjustments or for planning of the additions. It is also possible to move the process fluid from one point to another by pumping systems.

We have carried out the processing of slaughterhouse wastes (paunch and other fractions) together with molasses (US Patent Application (US20160251684A1) (Hakalehto 2016c)). In these cases the fructose of the molasses is converted into mannitol. When the molasses are added to the residual “zero fiber” fraction of the pulp and paper industries, this leads to the formation of organic acids, particularly lactic acid (Beckinghausen et al. 2019). Moreover, if paunch and molasses are added to this side stream, this also leads to the accumulation of mannitol in the favourable conditions in the multi-strain process.

In an advantageous mode of processing various wastes into mannitol and lactate, or into other organic acids, a mixed microbial culture of rumen bacteria can be used as the biocatalyst. This approach can be performed according to the procedure of the US Patent Application (US20160251684A1) (Hakalehto 2016c) These processes can be carried out simultaneously, namely the lactate and mannitol production, in the one and same reactor system. However, according to the present invention, the optimal process mode is a partially separated system of two pools (FIG. 1).

One important aspect is the difference in the composition of the LAB microflora. The flora in the lactate production phase (out of the hydrolyzed cellulose) have the optimal temperature of 28-32° C., whereas the mannitol production is carried out by strains selected at 35-40° C. The former process takes about 90-100 hours to reach maximal production rate, and the latter one about 50-70 hours for the same level.

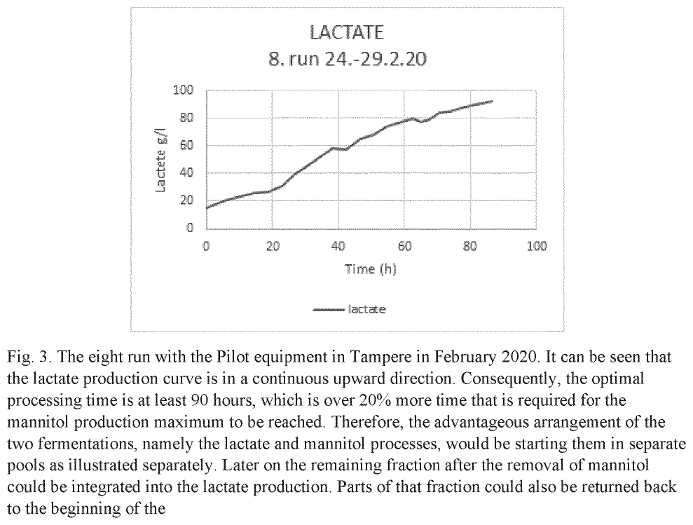

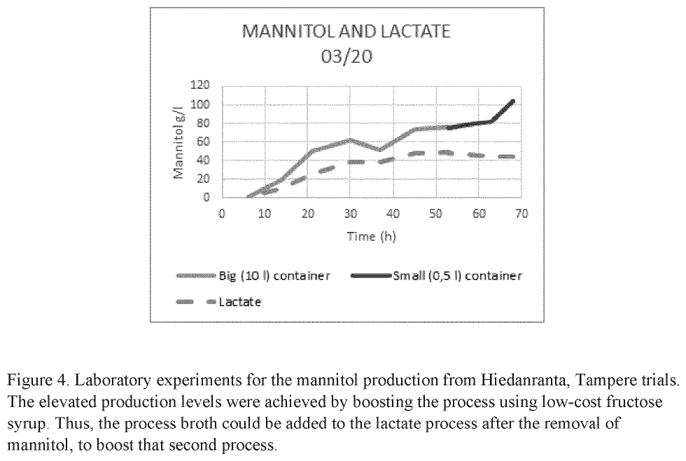

However, in the large-scale treatment of e.g. cellulosic waste combined with molasses, it turned out that the lactate process (FIG. 3) is at least 20 hours more time-consuming in reaching the metabolic completion than the mannitol process (FIG. 4). In this context the term “metabolic completion” refers to the maximal yields of the mixed fermentation. The two processes support each other:
1. The accumulation of lactate supports the mannitol process, as the lowering pH protects and preserves the product mannitol.
2. The mannitol process outcome is beneficial for the last stages of lactate production, as the residual fraction after the recovery of the product mannitol is combined with the final stages of the lactate production for boosting the production rate and product yield. Then it is possible to elevate the temperature from 28-32° C. to 35-40° C.

It is also noteworthy, that in lowered oxygen content, more butyric acid and hydrogen can be formed.

This synergism of two separated reaction is optimal and effective only when the processes are synchronized with the main processes starting in separate reactors or tanks or pools but to be combined in a delicate way as illustrated here (FIG. 1). Consequently, we developed and tested the method, by the teachings of the present invention by which the lactate fermentation and mannitol production are started in different reactors or pools, and then the broths of the two reactors are combined as instructed here. At this point, the mannitol is often preferably removed from the corresponding process fluid. It can also be added within the entire process fluid to the pool number 1, but then the total volume will increase very large. These reactions can be of the CBP-type with the enzymes still active in the broth.

After the mannitol production has reached its maximum, and the product recovered, for example by a separate reactor for crystallization, or by a series of reactors, the remaining active biological fluid can be added to the lactate production unit and into the lactate fermentation broth. There it can boost the lactate production. —During the mannitol production, the initial lactic acid bacteria (LAB) originating from the rumen contribute to the preservation of mannitol by keeping the pH low (Hakalehto 2016c). The division and initiation of the two processes in two reactors increase the production of both of the processes, as they can be adjusted and optimized separately for the beginning. However, it is advantageous to combine the residual fraction of the mannitol process into the ongoing lactate production, which brings also other synergistic benefits that can be achieved by this combination. Moreover, lactate is one of the main natural product of the rumen LAB, which, besides the stabilization of mannitol, also can be collected as a by-product from that process (pool number 2).

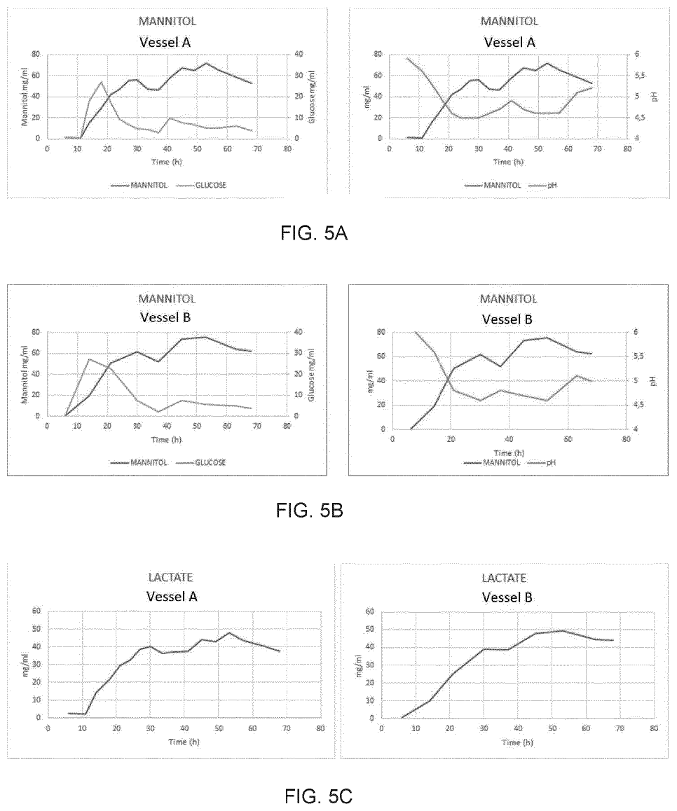

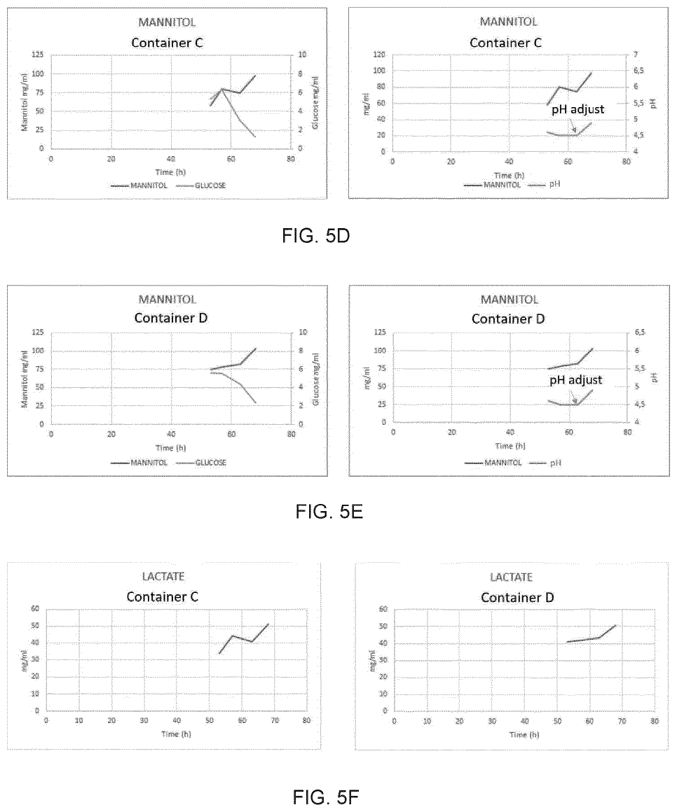

In order to boost mannitol production, the addition of fructose-containing substances into the containers served the purpose (FIG. 5. D.-E.). This improvement was clearly observable in comparison with Vessel A and B (FIG. 5. A.-B.). The lactate production was elevated in the end in the separated fermentation (container) (FIG. 5. F.). In the reactor, where both lactate and mannitol were produced in the same reactor pool, the production deceased in the end (FIG. 5.C.).

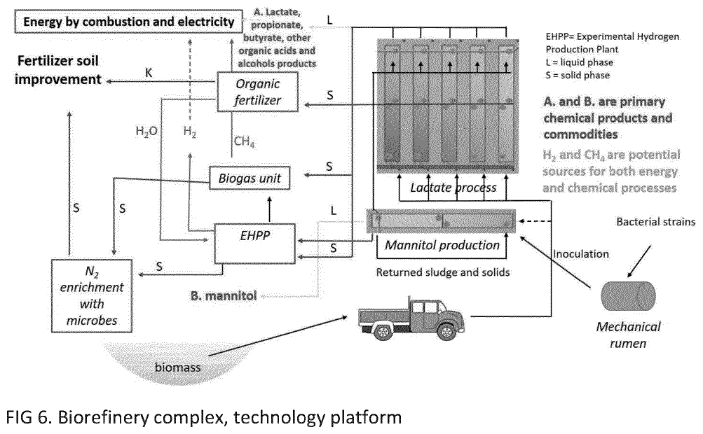

The production of such biochemicals serves as the core function in the conversion of biomass side streams into useful chemicals, energy gasses and organic fertilizers (FIG. 6). This plan for the production plant includes separated technical units, such as the lactate and mannitol pool reactors, hydrogen production unit, atmospheric nitrogen fixing for upgrading the organic fertilizer. mechanical rumen will be used for the production of the inoculum.

### Example 1

In the industrial piloting of lactate production from the zero fiber, 600 litres of the cellulolytic material was treated with 1000 g of Viscamyl Flow and 750 g of Optidex enzymes. The hydrolysis phase prior to the microbial process lasted for 25 hours. For the hydrolysis 300 litres of water was added, 50% of which was obtained from the residual fraction of the previous runs. For the microbial inoculum, 51 kg of rumen biomass and 7 kg of sour milk were added 20 hours after the onset of the fermentation phase. Also 175 kg of molasses were added, together with the microbes and 65 litres of NaOH (40%) and 17 kg of CaCO₃ for the pH adjustment during the process, as well as 21.5 kg of meat bone meal. The volume of the process water was increased by 127 litres during the process run. The pH was kept between 5.1-6.5 by the addition of NaOH, and the temperature was 30° C., which favoured the lactic acid bacteria derived from the lake. The steadily increasing lactate production is presented in FIG. 3. The results of this experiment indicated steady growth of lactate concentration during the experiment, reaching 9.2% in the end.

### Example 2

In the simultaneous production of mannitol and lactate in the laboratory, the focus was in the optimization of the former substance, since the optimization of the lactate as a product was carried out as described in the Example 1. The mannitol production was boosted for the last quarter of the process run by adding some fructose syrup to the broth. The temperature for the hydrolysis was 40° C., and it was 37° C. for the mannitol fermentation. Ten litre buckets were used as containers or reaction vessels.

The hydrolysis phase took 12 hours, and the enzymes “Viscamyl Flow” (2 g) and “Optidex” (1.4 g) were added to the suspension of 1.5 litres of zero fiber (or some corresponding cellulolytic substrate) with 0.5 litres of water. For the following microbial inoculation, 3.5 litres of rumen contents or paunch were added to the container together with 2.1 kg molasses, 500 g of meat bone meal and 100 g of liver. The pH adjustment during the process was carried out with 70 ml NaOH (40%) and 300 g CaCO₃, Up to 5 litres of water was added during the process. Regardless of the extensive dilution, the mannitol concentration reached 10.4% and lactate concentration elevated close to 5% without optimization. The hydrolysis can continue as the CBP reaction during the microbiological process.

In both Examples, the metabolites were monitored using NMR (Nucleic Magnetic Resonance) method (Laatikainen et al. 2016). These results indicated that after the completion of the mannitol process, the broth still contained glucose and mesophilic lactic acid bacteria. Their addition to the ongoing lactate fermentation in another reactor or pool could add the final yield particularly at the elevated temperature (30->37° C.).

In the mannitol process no more than 5% of the lactic acid was produced, whereas the production level in pool 1 was 9.2%. After the removal of mannitol, the residual fraction could induce higher lactate yields at 37° C. when added to pool 1 from pool 2. This could be deducted also from the relatively high level of glucose present in the broth according to the NMR (about 0.5%) in the end of the process. This indicates the potential of the microbial culture to elevate the lactate production during the remaining phase.

### REFERENCES

Beckinghausen, A., Dahlquist, E., Schwede, S., Lindroos, N., Retkin, R., Laatikainen, R., Hakalehto, E. (2019). Downstream processing of biorefined lactate from lake bottom zero fiber deposit—A techno-economic study on energy-efficient production of green chemicals. 11th International Conference of Applied Energy, Västerås 12-16 Aug. 2019.
Den Boer, E., Lucaszewska, A., Kluczkiewicz, D., Lewandowska, D., King, K., Reijonen, T., Suhonen, A., Jääskeläinen, A., Heitto, A., Laatikainen, R., Hakalehto, E. (2016). Volatile fatty acids as an added value from biowaste. Waste Management, 58: 62-69.
Hakalehto, E. (2008). Biotechnical and microbiological production method and equipment. European Patent Application 08701690.3.
Hakalehto, E. (2011). Simulation of enhanced growth and metabolism of intestinal Escherichia coli in the Portable Microbe Enrichment Unit (PMEU). In: Rogers M C, Peterson N D (eds.) E. coli infections: causes, treatment and prevention. New York, USA: Nova Science Publishers, pp. 159-175.
Hakalehto, E. (Ed.) (2012). Alimentary microbiome—a PMEU approach. New York, NY, USA: Nova Science Publishers, Inc.
Hakalehto, E. (2013). Interactions of Klebsiella sp. with other intestinal flora. In Pereira, L. A. & Santos, A. (eds.) Klebsiella infections: Epidemiology, pathogenesis and clinical outcomes. Nova Science Publishers, Inc. New York, USA.
Hakalehto, E. (2015a). Enhanced microbial process in the sustainable fuel production. In: Jinyue, Y (ed.). Handbook of clean energy systems. Wiley JR & Sons. Inc, Chichester, West Sussex, UK.
Hakalehto, E. (2015b). Hygienic lessons from the dairy microbiology cases. In: Hakalehto, E. (ed.) Microbiological food hygiene. New York, NY, USA: Nova Science Publishers, Inc.
Hakalehto, E. (2016a). Microbiological surveillance methods for the industries: comments on general strategies and theoretical background. In: Hakalehto, E. (ed.) Microbiological Industrial Hygiene. New York, NY, USA: Nova Science Publishers, Inc.
Hakalehto, E. (2016b). The many microbiomes. In: Hakalehto, E. (ed.) Microbiological Industrial Hygiene. New York, NY, USA: Nova Science Publishers, Inc.
Hakalehto, E. (2016c). Utilization of intestinal bacteria from slaughtered animals in industrial processes and waste treatment. US Patent Application US20160251684A1.
Hakalehto, E. (2018a). The effects of bioprocess scale, intracellular milieu and environmental parameters. In: Hakalehto, E. (ed.) Microbiological Environmental Hygiene. New York, NY, USA: Nova Science Publishers, Inc.
Hakalehto, E. (ed.) (2018b) Microbiological Environmental Hygiene. New York, NY, USA: Nova Science Publishers, Inc.
Hakalehto, E. (2020). Current megatrends in food production related to microbes. Journal of Food Chemistry and Nanotechnology 6(1): 78-87.
Hakalehto, E., Hänninen, 0. (2012). Gaseous CO2 signal initiate growth of butyric acid producing Clostridium butyricum both in pure culture and in mixed cultures with Lactobacillus brevis. Can J Microbiol, 58: 928-931.
Hakalehto, E., Jääskeläinen, A. (2017). Reuse and circulation of organic resources and mixed residues. In: Dahlquist, E. and Hellstrand, S. (Eds.) Natural resources available today and in the future: how to perform change management for achieving a sustainable world. Springer Verlag, Germany.
Hakalehto, E., Humppi, T., & Paakkanen, H. (2008). Dualistic acidic and neutral glucose fermentation balance in small intestine: Simulation in vitro. Pathophysiology, 15: 211-220.
Hakalehto, E., Heitto, A., Andersson, H., Lindmark, J., Jansson, J., Reijonen, T., Suhonen, A., Jääskeläinen, A., Laatikainen, R., Schwede, S., Klintenberg, P., Thorin, E. (2016). Some remarks on processing of slaughterhouse wastes from ecological chicken abattoir and farm. In: Hakalehto, E. (ed.) Microbiological industrial hygiene. New York, NY, USA: Nova Science Publishers, Inc.
Jääskeläinen, A., Rissanen, R., Jakorinne, A., Suhonen, A., Kuhmonen, T., Reijonen, T., Antikainen, E., Heitto, A., Hakalehto, E. (2016). How Does Modern Process Automation Understand the Principles of Microbiology and Nature. 9th EUROSIM Congress on Modelling and Simulation (EUROSIM 2016) 12-16 Sep. 2016 Oulu, Finland.
Laatikainen, R., Laatikainen, P., Hakalehto, E. (2016). Quantitative quantum mechanical nmr analysis: The superior tool for analysis of biofluids. In Proceedings of the 1st Int. Electron. Conf. Metabolomics, 1-30 Nov. 2016; Sciforum Electronic Conference Series, Vol. 1, 2016, C005; doi:10.3390/iecm-1-C005
Schwede, S., Thorin, E., Lindmark, J., Klintenberg, P., Jääskeläinen, A., Suhonen, A., Laatikainen, R., Hakalehto, E (2017). Using slaughterhouse waste in a biochemical based biorefinery-results from pilot scale tests. Environmental Technology, 38: 1275-1284.

1. Method for optimizing the simultaneous or interlinked production of (1) organic acids, such as lactate and (2) mannitol characterized in, that two reactor pools are used, which are the pool number 1 for the production of lactate or other SCFA's (Small Chain Fatty Acids), and the pool number 2 for the production of mannitol using rumen bacteria as biocatalysts in such a way that after the recovery of mannitol or even without it, the residual process fluid is advantageously applied to the pool number 1 for further elevating both the lactate and mannitol levels of the biorefining as a whole (FIG. 1).

2. Method according to the claim 1 characterized in, that the inoculation of the two reactor pools is carried out simultaneously.

3. Method according to the claim 1 characterized in, that zero fiber or other cellulosic material in the reactor 1 is used as the main source of glucose in the reactor pool.

4. Method according to the claim 3 characterized in, that cellulolytic enzymes are used for the hydrolysis of the cellulose, preferably at least partially in the CBP mode, simultaneously with the microbial processes.

5. Method according to the claim 1 characterized in, that fructose containing side streams were used as the raw material source for mannitol production.

6. Method according to the claim 1 characterized in, that the mannitol is recovered either from the pool 2 into which the fructose and rumen bacteria had been added, or from the pool 1 if the residues of the pool 2 are transferred to pool 1 without prior recovery of the mannitol.

7. Method according to any of the claims 1-6 characterized in, that some kind of cellulosic material such as the zero fiber, was used in both of the pools number 1 and 2 amongst the other raw materials.

8. Method according to the claim 1 characterized in, that the purification of lactate or other organic acids can be carried out of the residues of both pools either separately or as combined to each other.

9. Method according to the claim 1 characterized in, that hydrogen in the bubble flow (FIG. 2) is collected by suction for further use as an energy gas or a reducing agent.

10. Method according to the claim 1 characterized in, that the final fraction with solid particles or suspension is collected for soil improvement or organic fertilization purposes.

11. Method according to the claim 10 characterized in, that the residual fraction of the biorefinery is upgraded as soil improvement by using bacteria of the species Clostridium pasteurianum or any other autonomously nitrogen-fixing species for increasing the soil nitrogen content available for the plant growth.

12. Method according to the claim 10 characterized in, that the final fraction is used for replacing or increasing the soil humic fraction,

13. Apparatus for using the method as described in the claim 1 characterized in, that the two pools are advantageously arranged conveniently into such a position with respect to each other that the residues of the pool 2 can be added the shortest way into the pool 1 in the process phase that corresponds to the time point in the latter pool and process (X point in FIG. 1).

14. Apparatus according to the claim 13 characterized in, that the process fluid or flow or broth or suspension is moving forwards from the beginning to the end of the process by the help of rotors, screws, blows, paddlewheels or equivalent.

15. Apparatus according to the claim 13 characterized in, that sensors or other measurement systems for temperature, pH, turbidity, contents of various gases, conductivity, pO₂, pCO₂, impedance, viscosity, glucose or fructose content, or any other relevant or measurable parameter for the bioprocess can be situated at any point of the process or process flow in any of the pools.

16. Apparatus according to the claim 13 characterized in, that the process can be adjusted in any of the pools with respect to the chosen parameters at any time point during the process flow; for example at the temperature of 28-32° C. for the lactate-producing LAB population in the pool 1, and at 37-42° C. for the corresponding population in the pool 2.

17. Apparatus according to the claim 13 characterized in, that the process control and adjustment or addition of reagents or water is being facilitated by the results of the measurements.

18. Apparatus according to the claim 13 characterized in, that the mannitol is recovered by crystallization or by any other method carried out in a separate container or series of containers from the fluid of the pool 2.

19. Apparatus according to the claim 13 characterized in, that the lactate is the main product in pool 1, whereas it is the additional product in pool 2, but the same equipment can be used for its recovery in both cases.
